(19) United States
(12) Patent Application Publication
(10) Pub. No.: US 2023/0159869 A1
(43) Pub. Date: May 25, 2023

(54) DEVICE AND METHOD FOR ACHIEVING RNAI OF INSECTS BY ATOMIZATION

(71) Applicant: Qingdao Agricultural University, Qingdao (CN)

(72) Inventors: Chunhong YANG, Qingdao (CN); Qingfeng ZHANG, Qingdao (CN); Tongxian LIU, Qingdao (CN)

(21) Appl. No.: 17/871,387

(22) Filed: Jul. 22, 2022

(30) Foreign Application Priority Data
Nov. 20, 2021 (CN) 202111380961.6

(51) Int. Cl. C12M 1/04

(52) U.S. Cl. CPC C12M 1/04

(57) ABSTRACT

A device and method for achieving RNAi of insects by atomization are provided. The device includes an insect placing box installed on a chemical solution atomizer; a power plug is provided at one side of the chemical solution atomizer, an atomization release mechanism is provided in the chemical solution atomizer and is in communication with the insect placing box. The atomization release mechanism includes an atomized gas release port formed on the insect placing box, an ultrasonic atomizer arranged below the atomized gas release port, a suction tube, a chemical solution storage chamber connected to an end of the suction tube, and a piston installed on the chemical solution storage chamber. One sidewall surface of the chemical solution atomizer is an operation panel suitable for controlling atomization. An atomization regulating switch is installed on the operation panel.

### CROSS REFERENCE TO RELATED APPLICATION

This patent application claims the benefit and priority of Chinese Patent Application No. 202111380961.6 filed on 20 Nov. 2021, the disclosure of which is incorporated by reference herein in its entirety as part of the present application.

### TECHNICAL FIELD

The present disclosure relates to the technical field of interference experiments of insects, and in particular relates to a device and method for achieving RNAi of insects by atomization.

### BACKGROUND ART

In RNAi interference (RNA (Ribonucleic Acid) interference) experiments of insects, the chemical delivery to insects is achieved by methods, such as micro-injection, feeding, and soaking. The micro-injection method refers to injecting synthetic dsRNA (double-stranded RNA) into the bodies of insects by an injection method, which can prevent the barrier effect of epidermis and intestine of the insect from delivering dsRNA to target cells precisely. The feeding method refers to feeding dsRNA to insects making the dsRNA enter the bodies of the insects. The soaking method refers to soaking insects in a dsRNA solution to enable the dsRNA solution to enter the body of the insect through the body wall. The dsRNA will attenuate or even block the expression of the target gene, thus achieving the experimental effect.

However, in the above methods, the micro-injection method can cause damage to the insects and is very difficult to operate and unsuitable for insect experiments having a large number of insects. The feeding method enables the chemical to pass through the digestive system, thus the chemical function can be hindered by the barrier effect of the intestinal tract. The soaking method may seal spiracles of the insects and affect the respiration of the insects, leading to the death of the insects. Therefore, there is an urgent need to research and develop a chemical delivery device which does not damage the insect bodies and can meet requirements such as the number of the insects in the experiment. To this end, a solution is generated through deep research on the above problems.

### SUMMARY

In order to solve the above problems, an objective of the present disclosure is to design a device and method for achieving RNAi of insects by atomization to solve the problems in the existing background art.

The technical solution of the present disclosure for achieving the objective above is as follows: a device for achieving RNAi of insects by atomization includes an insect placing box and a chemical solution atomizer. The insect placing box is a box of a rectangular structure and is installed on the chemical solution atomizer; a power plug is provided at one side of the chemical solution atomizer, an atomization release mechanism is provided in the chemical solution atomizer to be in communication with the interior of the insect placing box, and a clamping assembly is provided between the insect placing box and the chemical solution atomizer.

The atomization release mechanism includes an atomized gas release port, an ultrasonic atomizer, a suction tube, a chemical solution storage chamber, and a piston. The atomized gas release port is formed on the bottom surface of the insect placing box, the ultrasonic atomizer is correspondingly arranged below the atomized gas release port, the ultrasonic atomizer is connected to the below suction tube, the chemical solution storage chamber is connected to an end of the suction tube, and the piston is installed on the chemical solution storage chamber.

One sidewall surface of the chemical solution atomizer is an operation panel suitable for controlling atomization.

An atomization regulating switch is installed on the operation panel.

The clamping assembly may include a pair of clamping grooves and a pair of protrusions.

The pair of clamping grooves are arranged at both sides of the bottom surface of the insect placing box, the pair of protrusions engage with the clamping grooves and are arranged at both sides of the top surfaces of the chemical solution atomizer; each clamping groove is a groove of a rectangular structure, and each protrusion capable of engaging with the clamping groove is a batten of a rectangular structure protruding from the top surface of the chemical solution atomizer.

A hygrometer is provided in the insect placing box, and the operation panel of the chemical solution atomizer in communication with the insect placing box is provided with a humidity setting button.

The operation panel is provided with a display screen, and a warning light and a power light are sequentially arranged below the display screen.

A delivery tube is provided on one side of the chemical solution storage chamber, the delivery tube penetrates through a side wall of the chemical solution atomizer, and the side wall of the chemical solution atomizer is provided with a chemical solution inlet to be in communication with the delivery tube.

The insect placing box is a box of a rectangular structure with an opening at its top.

A method for achieving RNAi of insects by atomization includes the following steps: step S1, marking and placing insects into a box; step S2, collecting and setting air humidity; step S3; preparing an atomization solution; step S4, atomizing the atomization solution; step S5, observing and recording; and step S6, cleaning an experimental device;

step S1: marking and grouping the insects for testing, distinguishing and marking the insects according to different interference experiment standards, and placing the marked insects into an insect placing box;

step S2: collecting humidity by a hygrometer in the insect placing box, and setting humidity after atomization according to the collected humidity;

step S3: preparing the atomization solution for RNAi as required, and adding the atomization solution into the chemical solution storage chamber through a chemical solution inlet;

step S4: regulating an atomization regulating switch, and controlling a piston to push the atomization solution to enter an ultrasonic atomizer for atomization according to experimental demands, and then releasing atomized gas through an atomized gas release port into the insect placing box;

step S5: allowing the insect placing box to sit for a period of time, and taking out the insects for determining genetic expression of dsRNA; and

step S6: removing the insect placing box from the chemical solution atomizer for cleaning, and injecting clear water into the chemical solution storage chamber for atomization cleaning.

In the step S3, the chemical solution is injected by inserting a syringe into the chemical solution inlet.

In the step S4, the atomization concentration can be displayed by a display screen.

In the step S5, the sitting time is 4-8 hours, and is adjusted according to the concentration of the atomization solution for each experiment.

The device for achieving RNAi of insects by atomization manufactured by using the technical solution of the present disclosure is simple in structure and convenient to control. By adopting a mode of atomizing and spraying the chemical, the influence of the RNAi achieved by atomization on the insects is smaller, operation is more convenient, the chemical can be delivered to a plurality of insects in a large range, the spraying chemical is uniform, the concentration of the chemical can be measured and regulated. A method for performing chemical delivery by atomization according to the present device is simple in step, convenient to operate, minimizes or eliminates damage to insect bodies, and is high in experimental efficiency.

### BRIEF DESCRIPTION OF THE DRAWINGS

FIG. 1 is a schematic axonometric view of a structure of a device for achieving RNAi of insects by atomization of the present disclosure;

FIG. 2 is a schematic front view of a structure of a device for achieving RNAi of insects by atomization of the present disclosure;

FIG. 3 is a schematic side view of a structure of a device for achieving RNAi of insects by atomization of the present disclosure;

In the drawings: 1 is insect placing box; 2 is chemical solution atomizer; 3 is atomized gas release port; 4 is ultrasonic atomizer; 5 is suction tube; 6 is chemical solution storage chamber; 7 is piston; 8 is atomization regulating switch; 9 is hygrometer; 10 is humidity setting button; 11 is display screen; 12 s warning light; 13 is power light; 14 is delivery tube; 15 is chemical solution inlet; 16 is power plug; 101 is clamping groove; 201 is protrusion.

### DETAILED DESCRIPTION OF THE EMBODIMENTS

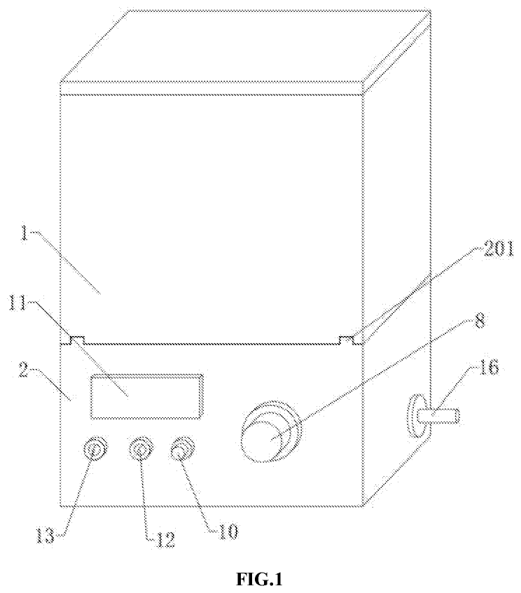

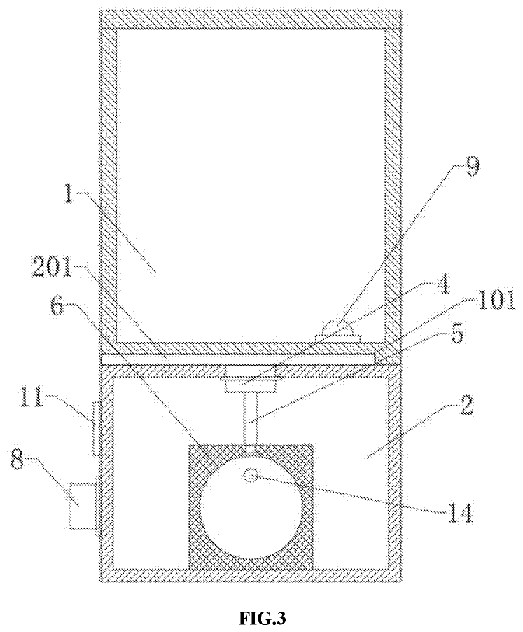

The following specifically describes the present disclosure in detail with reference to the accompanying drawings. As shown in FIGS. 1 to 3, the present disclosure relates to a device comprising an insect placing box and a chemical solution atomizer, the insect placing box is a box of a rectangular structure. The insect placing box is installed on the chemical solution atomizer, and a power plug is provided on one side of the chemical solution atomizer, an atomization release mechanism is arranged in the chemical solution atomizer to be in communication with the interior of the insect placing box, and a clamping assembly is provided between the insect placing box and the chemical solution atomizer. The atomization release mechanism comprises an atomized gas release port, an ultrasonic atomizer, a suction tube, a chemical solution storage chamber, and a piston. The atomized gas release port is provided at the bottom surface of the insect placing box, the ultrasonic atomizer is correspondingly arranged below the atomized gas release port, the ultrasonic atomizer is connected to the below suction tube, the chemical solution storage chamber is connected to an end of the suction tube, and the piston is installed on the chemical solution storage chamber. One sidewall surface of the chemical solution atomizer is an operation panel suitable for controlling atomization. An atomization regulating switch is installed on the operation panel. The present device for achieving RNAi of insects by atomization is simple in structure and convenient to control. By adopting a mode of atomizing and spraying the chemical, the influence of the RNAi achieved by atomization on the insects is smaller, operation is more convenient, the chemical can be delivered to a plurality of insects in a large range, the spraying chemical is uniform, and the concentration of the chemical can be measured and regulated. A method for performing chemical delivery by atomization according to the present device is simple in step, convenient to operate, minimizes or eliminates damage to insect bodies, and is high in experimental efficiency.

By those skilled in the art, all electric elements in this case are connected to power supplies adapted to the electric elements by wires; a proper controller can be selected according to actual conditions so as to meet control demands. The specific connection and control sequence should refer to the working sequences among various electric elements in the following working principle so as to complete the electrical connection. The connection means are well known in the art, and the following mainly describes the working principle and process rather than describing the electric control.

Embodiment: as shown in FIGS. 1 to 3 of the description, a device for achieving RNAi of insects by atomization is provided, the device comprises an insect placing box 1 and a chemical solution atomizer 2. The insect placing box 1 is a box of a rectangular structure, the insect placing box 1 is installed on the chemical solution atomizer 2, a power plug 16 is provided on one side of the chemical solution atomizer 2, an atomization release mechanism is arranged in the chemical solution atomizer 2 to be in communication with the interior of the insect placing box 1, and a clamping assembly is arranged between the insect placing box 1 and the chemical solution atomizer 2. During specific implementation, the insect placing box 1 is a main vessel for placing insects in the experiment as well as a space for atomized gas release in the experiment. Before the experiment, the insects are placed into the insect placing box 1, the chemical solution atomizer 2 is used for atomizing chemical solution into atomized gas and then introducing the atomized gas into the insect placing box 1 for an atomization testing. The insect placing box 1 acts as the general control of the device. The clamping assembly is configured for being tightly connected to the insect placing box 1 and the chemical solution atomizer 2.

As shown in FIGS. 1 to 3 of the description, the atomization release mechanism comprises an atomized gas release port 3, an ultrasonic atomizer 4, a suction tube 5, a chemical solution storage chamber 6, and a piston 7, a connection relation and a positional relation of which are as follows:

The atomized gas release port 3 is provided on the bottom surface of the insect placing box 1, the ultrasonic atomizer 4 is correspondingly arranged below the atomized gas release port 3, the ultrasonic atomizer 4 is connected to the below suction tube 5, the chemical solution storage chamber 6 is connected to an end of the suction tube 5, and the piston 7 is installed on the chemical solution storage chamber 6.

One sidewall surface of the chemical solution atomizer 2 is an operation panel suitable for controlling atomization.

An atomization regulating switch 8 is installed on the operation panel.

During specific implementation, after the insect placing box 1 is installed on the chemical solution atomizer 2, the atomized gas release port 3 at the bottom of the insect placing box 1 is flush with a nozzle of the ultrasonic atomizer 4 at the top surface of the chemical solution atomizer 2, the chemical solution is stored in the chemical solution storage chamber 6, and the atomization regulating switch 8 is regulated to control the pushing speed and the pushing stroke of the piston 7, such that the piston 7 pushes the chemical solution to enter the ultrasonic atomizer 4 via the suction tube 5, and then the chemical solution is introduced into the atomizer release port 3 after being atomized by the ultrasonic atomizer 4.

As shown from FIGS. 1 to 3 of the description, the clamping assembly comprises a pair of clamping grooves 101 and a pair of protrusions 201, a connection relation and a positional relation of which are as follows:

the pair of clamping grooves 101 are provided at both sides of the bottom surface of the insect placing box 1, and the pair of protrusions 201 capable of engaging with the clamping grooves 101 are provided at both sides of the top surface of the chemical solution atomizer 2. Each of the clamping grooves 101 is a groove of a rectangular structure, and each protrusion 201 capable of engaging the clamping groove is a batten of a rectangular structure protruding from the top surface of the chemical solution atomizer 2.

During the specific implementation, the clamping grooves 101 correspond to the protrusions 201 during installation, the insect placing box 1 is pushed onto the chemical solution atomizer 2, thus increasing the tightness and stability of the connection.

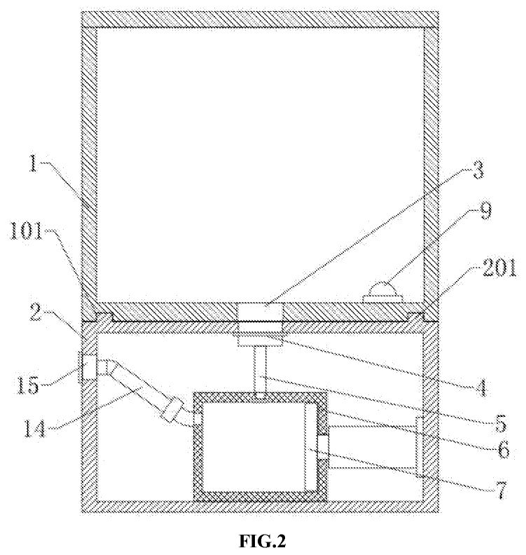

As shown in FIGS. 1 and 2, a hydrometer 9 is provided in the insect placing box 1, an operation panel of the chemical solution atomizer in communication with the insect placing box is provided with a humidity setting button 10, and after measuring the humidity, the atomization concentration of the chemical solution is determined by the set humidity value.

As shown in FIG. 1, the operation panel is provided with a display screen 11, a warning light 12 and a power light 13 are sequentially arranged below the display screen 11, the display screen 11 is configured for displaying the atomization concentration, the power light 13 is configured for displaying a power-on state, and the warning light 12 displays that the atomization concentration of the device exceeds the standard requirement.

As shown in FIG. 12, a delivery tube 14 is provided on one side of the chemical solution storage chamber 6, the delivery tube 14 penetrates through a side wall of the chemical solution atomizer 2, the side wall of the chemical solution atomizer 2 is provided with a chemical solution inlet 15 to be in communication with the delivery tube, and the chemical solution inlet 15 is configured for adding chemical solution into the chemical solution storage chamber 6.

The insect placing box 1 is a box of a rectangular structure with an opening at its top, and the insects are placed into the box from the upper part.

After the above experimental device is prepared, an experiment for achieving RNAi of insects by atomization is carried out according to the following methods:

step S1: the insects for testing are grouped and marked, the insects are distinguished and marked according to different interference experiment standards, and the marked insects are placed into an insect placing box 1;

step S2: humidity is collected by using a hygrometer 9 in the insect placing box 1, and the humidity is set after atomization according to the collected humidity;

step S3: an atomization solution for RNAi is prepared as required, and the atomization solution is added into the chemical solution storage chamber 6 from a chemical solution inlet 15, wherein the chemical solution is injected by inserting a syringe into the chemical solution inlet 15;

step S4: an atomization regulating switch 8 is regulated, and a piston 7 is controlled to push the atomization solution to enter an ultrasonic atomizer 4 for atomization according to experimental demands, and then the atomized gas is released from an atomized gas release port 3 into the insect placing box 1, wherein the atomization concentration can be displayed by a display screen 11;

step S5: the device is allowed to sit for a period of time, and the insects are taken out of the insect placing box 1 for determining genetic expression of dsRNA; and

step S6: the insect placing box 1 is removed from the chemical solution atomizer 2 for cleaning, and clean water is injected into the chemical solution storage chamber 6 for atomization cleaning, wherein the sitting time is 4 to 8 hours, and is adjusted according to the concentration of the atomization solution for each test.

In conclusion, the present device for achieving RNAi of insects by atomization is simple in structure and convenient to control. By adopting a mode of atomizing and spraying chemical, the influence of the RNAi achieved by atomization on the insects is smaller, operation is more convenient, the chemical can be delivered to a plurality of insects in a large range, the spraying chemical is uniform, and the concentration of the chemical can be measured and regulated. The method for performing chemical delivery by atomization according to the present device is simple in step, convenient to operate, minimizes or eliminates damage to insect bodies, and is high in experimental efficiency.

The above technical solution only embodies the preferred technical solution of the technical solutions of the present disclosure, and some variations which may be made by a person skilled in the art to some parts of the technical solution embody the principle of the present disclosure and fall within the scope of protection of the present disclosure.

What is claimed is:

1. A device for achieving RNAi of insects by atomization, comprising an insect placing box and a chemical solution atomizer, wherein the insect placing box is a box of a rectangular structure and is installed on the chemical solution atomizer; a power plug is provided at one side of the chemical solution atomizer, an atomization release mechanism is provided in the chemical solution atomizer and is in communication with an interior of the insect placing box, and a clamping assembly is provided between the insect placing box and the chemical solution atomizer;
one sidewall surface of the chemical solution atomizer is an operation panel suitable for controlling atomization; and
an atomization regulating switch is installed on the operation panel.

2. The device for achieving RNAi of insects by atomization according to claim 1, wherein a hygrometer is provided inside the insect placing box.

3. The device for achieving RNAi of insects by atomization according to claim 1, wherein the operation panel is provided with a display screen.

4. The device for achieving RNAi of insects by atomization according to claim 1, wherein a delivery tube is provided on one side of a chemical solution storage chamber.

5. The device for achieving RNAi of insects by atomization according to claim 1, wherein the insect placing box is a rectangular-structure box with an opening at its top.

6. A method for achieving RNAi of insects by atomization, comprising:
step S1, marking and placing insects into an insect placing box; step S2, collecting and setting air humidity; step S3, preparing an atomization solution; step S4, atomizing the atomization solution; step S5, observing and recording; and step S6, cleaning an experimental device;
in the step S1: marking and grouping the insects for testing, distinguishing and marking the insects according to different interference experiment standards, and placing the marked insects into the insect placing box;
in the step S2: collecting humidity by a hygrometer in the insect placing box, and setting humidity after atomization according to the collected humidity;
in the step S3: preparing the atomization solution for RNAi as required, and adding the atomization solution into a chemical solution storage chamber through a chemical solution inlet;
in the step S4: regulating an atomization regulating switch, and controlling a piston to push the atomization solution to enter an ultrasonic atomizer for atomization according to experimental demands, and then releasing atomized gas through an atomized gas release port into the insect placing box;
in the step S5: allowing the insect placing box to sit for a time, and taking out the insects for determining genetic expression of dsRNA; and
in the step S6: removing the insect placing box from the chemical solution atomizer for cleaning, and injecting clean water into the chemical solution storage chamber for atomization cleaning.

7. The method for achieving RNAi of insects by atomization according to claim 6, wherein in the step S3, the chemical solution is injected by inserting a syringe into the chemical solution inlet.

8. The method for achieving RNAi of insects by atomization according to claim 6, wherein in the step S4, an atomization concentration is displayed by a display screen.

9. The method for achieving RNAi of insects by atomization according to claim 6, wherein in the step S5, the sitting time is 4-8 hours, and is adjusted according to concentration of the atomization solution for experiment.
